(19) United States
(12) Patent Application Publication
(10) Pub. No.: US 2014/0012114 A1
(43) Pub. Date: Jan. 9, 2014

(54) Monitoring of Fluid Content

(71) Applicants: Nederlandse Organisatie voor Toegepast-Natuurwetenschappelijk Onderzoek - TNO, Delft (NL); Stichting IMEC Nederland, Eindhoven (NL)

(72) Inventors: Marcel Zevenbergen, Rotterdam (NL); Lindsay Brown, Mierlo (NL); Maria Peter, Eindhoven (NL); Mercedes Crego Calama, Geldrop-Mierlo (NL)

(21) Appl. No.: 13/936,114

(22) Filed: Jul. 5, 2013

(30) Foreign Application Priority Data
Jul. 6, 2012 (EP) 12175473.3

(51) Int. Cl. A61B 5/145

(52) U.S. Cl. CPC A61B 5/14521
USPC 600/346

(57) ABSTRACT

This application describes methods and apparatus for monitoring of fluid content that are suitable for in-situ, real time and/or continuous monitoring, especially of bodily fluids and in particular the content of sweat. The application also describes fabrication of such an apparatus. The apparatus comprises a multilayer structure comprising at least two electrode layers for detection of fluid content separated by at least one insulating layer. The multilayer structure defines at least one flow channel which provides a flow path for continuous flow of fluid in use, and the electrode layers form part of the sidewall of the flow channel(s). The flow channel(s) may run in a direction substantially perpendicular to the layers. The electrode layers may employ electrochemical detection and may comprise a reference electrode and an ion-sensitive electrode. The apparatus may be fabricated using various printing or deposition techniques.

### CROSS-REFERENCE TO RELATED APPLICATION

This application claims priority to European Patent Application Serial No. EP 12175473.3 filed Jul. 6, 2012, the contents of which are hereby incorporated by reference.

### BACKGROUND

A subject's sweat can contain useful information about the physiological condition of the subject. For example, it has been shown that both pH and chloride concentration in sweat rise significantly when a subject becomes dehydrated. As another example, more frequent muscle cramps have been observed in athletes who have had a large sodium loss during sweating and lactate levels have been shown to rise when muscles become exhausted.

Accordingly, sweat monitoring may be used to monitor the condition of a subject. For example, sweat monitoring may be used to alert a subject of possible dehydration. As another example, because the content of sweat may also be affected by drug use, sweat monitoring could be also useful in detecting drug abuse or use of prescribed substances in sports. Sweat monitoring has been used in the diagnosis and/or monitoring of a range of other medical conditions as well.

Typical sweat monitoring methods involve collecting a sample of sweat from a subject for subsequent analysis. In some applications, the subject may be provided with a sweat patch that is secured to the skin so as to collect and store sweat. The patch can then be later removed for subsequent analysis.

Other bodily fluids, such as saliva, may also provide useful information about the condition of a subject, in much the same manner as that described above for sweat.

### SUMMARY

One drawback of typical methods of sweat monitoring is that such methods allow only periodic analysis of sweat. These methods do not allow for continuous and/or real-time (in-situ) monitoring of sweat.

Disclosed are apparatuses for continuous and/or real-time (in-situ) monitoring of sweat. Also disclosed are methods for fabricating such apparatuses.

According to an exemplary embodiment, there is provided an apparatus for continuous monitoring of fluid content comprising: a multilayer structure comprising at least two electrode layers for detection of fluid content, said electrode layers being separated by at least one insulating layer, wherein the multilayer structure defines at least one flow channel, said at least one flow channel providing a flow path for continuous flow of fluid in use, and wherein said electrode layers form part of the sidewall of said at least one flow channel.

In some embodiments, the flow channel runs in a direction substantially perpendicular to the layers.

In some embodiments, one of the electrode layers may comprise a reference electrode layer which comprises an electrode in contact with a first material, wherein the first material forms said part of the sidewall of the flow channel and provides a substantially constant ionic concentration in use.

In some embodiments, at least one electrode layer may comprise an ion-selective electrode layer. In one embodiment a plurality of the electrode layers comprise ion-selective electrode layers. At least some of said ion-selective electrode layers may be sensitive to different target ionic species to one another. At least one ion-selective electrode layer may comprise an electrode formed from an electrode material sensitive to a target ionic species and said electrode material forms said part of the sidewall of the flow channel. Additionally or alternatively at least one ion-selective electrode layer may comprise an electrode in contact with an ion-selective membrane material wherein said ion-selective membrane material forms said part of the sidewall of the flow channel.

In some embodiments, the multilayer structure may further comprise at least one reactive layer forming part of the sidewall of the flow channel downstream of at least one of the electrode layers. The reactive layer may be configured to react with at least one target chemical, if present, to produce an electrochemically active product. At least one reactive layer may comprise a porous material having an enzyme immobilized therein that reacts with a target chemical.

In some embodiments, the multilayer apparatus may further comprise at least one sensing layer having a porous material in contact with at least one electrode treated with a sensing material that reacts with a target chemical, wherein the porous material forms part of the sidewall of the flow channel.

In some embodiments, there may be an absorbent material disposed at one end of the flow channel to draw fluid through the flow channel in use.

In some embodiments, the multilayer structure may comprise a plurality of flow channels, each flow channel having a plurality of electrode layers forming part of the sidewall of the flow channel, wherein the electrode layers vary between at least some flow channels to provide different sensing functionality within the flow channels.

In some embodiments, the apparatus may comprise circuitry for measuring the voltage and/or current at/or between one or more of the electrode layers.

In some embodiments, the apparatus may, in particular be a sweat monitoring apparatus where at least one flow channel provides a flow path for continuous flow of sweat in use. When used as a sweat monitoring apparatus there may be at least one layer of material capable of inducing sweating.

In some embodiments, the apparatus may also be used to monitor other bodily fluids and thus may relate to a bodily fluid sensor. The bodily fluid sensor may be contactable with a surface of the body such that the flow channel provides a flow path for fluid produced at that surface. Alternatively the apparatus may be arranged to be immersed in a fluid of interest or be manipulated into contact with a fluid of interest.

The description also relates to a fluid monitoring system comprising an apparatus for continuous monitoring of fluid content as described above and electrical circuitry. The electrical circuitry may include readout circuitry for readout of sensing data from said apparatus for continuous monitoring and circuitry for at least one of: storage of said data, transmission of said data and wireless communication. The circuitry may therefore comprise a suitable memory circuit, a transmission interface and/or a wireless communication module and possibly an antenna.

The description also relates to methods of manufacture of fluid monitoring apparatus. Thus in another aspect of the present description, there is provided a method of fabricating an apparatus for continuous monitoring of fluid content comprising: taking a substrate; forming a plurality of electrode layers over at least part of said substrate, successive electrode layers being separated by at least one insulating layer; and forming at least one channel through said layers and said substrate to define a flow path such that said electrode layers form part of the sidewall of said at least one channel.

The at least one channel may, in some embodiments, be formed in a direction substantially perpendicular to the layers.

In some embodiments, the method may comprise forming at least one reference electrode layer by forming an electrode in contact with a first material, wherein the first material provides a substantially constant ionic concentration in use; the area of first material does not wholly overlap with the area of the electrode; and the step of forming the at least one channel comprises forming said channel so that said first material forms part of the sidewall but the electrode itself does not form part of the sidewall.

In some embodiments, the at least one electrode layer may comprise an ion-selective electrode layer. A plurality of the electrode layers may comprise ion-selective electrode layers and at least some of the ion-selective electrode layers may be sensitive to different target ionic species to one another. In some embodiments, forming at least one ion-selective electrode layer may comprise forming an electrode from an electrode material sensitive to a target ionic species and the step of forming the at least one channel may comprise forming the channel so that said electrode material forms said part of the sidewall of the flow channel.

In some embodiments, forming at least one ion-selective electrode layer may comprise forming an electrode in contact with ion-selective membrane material wherein: the area of ion-selective membrane material does not wholly overlap with the area of the electrode; and the step of forming the at least one channel comprises forming said channel so that said ion-selective membrane material forms part of the sidewall but the electrode itself does not form part of the sidewall.

### BRIEF DESCRIPTION OF THE DRAWINGS

The disclosure will now be described by way of example only, with reference to the accompanying drawings, of which:

FIG. 1 illustrates an example monitoring apparatus, in accordance with some embodiments;

FIGS. 2A-E illustrate a method of fabricating a monitoring apparatus, in accordance with some embodiments;

FIGS. 3A-C illustrate another example method of fabricating a monitoring apparatus, in accordance with some embodiments;

FIG. 4 illustrates an example monitoring apparatus including a plurality of different ion-sensitive electrodes, in accordance with some embodiments;

FIG. 5 illustrates a an example monitoring apparatus including an ion-sensitive electrode layer having an electrode and an ion-selective membrane, in accordance with some embodiments;

FIG. 6 illustrates an example monitoring apparatus including a reactive layer and sensing electrodes, in accordance with some embodiments;

FIG. 7 illustrates an example monitoring apparatus having multiple sensing capability, in accordance with some embodiments;

FIG. 8 illustrates an example monitoring apparatus including a layer for inducing sweating, in accordance with some embodiments;

FIG. 9 illustrates an example monitoring apparatus including a sensing layer that reacts with target chemicals to provide a detectable change in physical properties, in accordance with some embodiments; and

FIG. 10 illustrates an example monitoring apparatus suitable for being immersed in or brought into contact with a fluid, in accordance with some embodiments.

### DETAILED DESCRIPTION

Disclosed are example apparatuses configured for the monitoring of fluids, including bodily fluids, such as sweat and saliva. Other fluids are possible as well. The example monitoring apparatuses may be particularly suitable for the monitoring of sweat, though the monitoring of other fluids using the example apparatuses is possible as well.

The example monitoring apparatuses may allow for substantially continuous and/or real time (in-situ) monitoring of the content of such fluids. To this end, the example monitoring apparatuses may use electrochemical detection principles to monitor for the presence of one or more analytes in a fluid. As described below, in some embodiments, a variety of different sensing electrode layers may be used in order to provide detection capability for a number of different target species.

In some embodiments, an example monitoring apparatus may include a multilayer structure. The multilayer structure may include at least two electrode layers, each of which is configured to detect a fluid content of a fluid. The multilayer structure may further include at least one insulating layer, and the at least two electrode layers may be separated from one another by the at least one insulating layer.

The multilayer structure may define at least one flow channel that provides a flow path for continuous flow of the fluid. The at least two electrode layers may be arranged to form part of a sidewall of the flow channel(s), so that the at least two electrode layers may detect the fluid content of the as it flows through the flow channel(s).

In embodiments where the example monitoring apparatus is configured for a bodily fluid (e.g., sweat), the example monitoring apparatus may be configured to contact a subject's body, with the flow channel providing a flow path for the fluid from the body.

In some embodiments, the multilayer structure may be fabricated as a relatively thin structure that can be applied to a subject as a patch in contact with the subject's body (e.g., a sweat patch for monitoring sweat content). The flow channel(s) may provide a flow path to a surface of the structure that, in use, is exposed to air and/or an absorbent material. The flow channel may therefore provide a flow path for fluid, such as sweat, through the structure to the atmosphere where it can evaporate. The continual evaporation of fluid from the surface of the patch will allow a continuous flow of fluid through the apparatus and past the sensing electrodes. Alternatively or additionally, in some embodiments the flow path may be in fluid contact with an absorbent material that absorbs the relevant fluid (e.g., sweat), thereby resulting in a continual drawing of fluid from the subject's body through the flow channel(s). In this way the fluid may be constantly replenished in the vicinity of the sensing electrodes without requiring any form of microfluidic system that would significantly add to the cost and complexity of the sensor. This allows the patch to be left in-situ to provide substantially continuous monitoring, allowing anaylsis of the fluid to occur in real time.

To facilitate analysis of the fluid, the sensing electrodes of the monitoring apparatus may be connectable to suitable readout circuitry. In some embodiments, the data acquired could be stored for later analysis which would provide a more complete record of the variation in the fluid content over time than previously possible with conventional monitoring apparatuses. Alternatively, in other embodiments the data may be communicated to a user to provide real-time feedback and/or analysis. The data may be communicated to some data processing circuitry which may be part of the monitoring apparatus or part of some other device located on the subject and/or the data may be communicated wirelessly to some remote monitoring device and thus the monitoring apparatus may be provided with wireless electronic readout and data transmission circuitry.

As mentioned above, the monitoring apparatus may operate using the principles of electrochemical detection. For example, the monitoring apparatus may be configured to determine the concentration of certain ions in the fluid. To this end, the multilayer structure may include one or more ion-selective electrodes (which, as described below, may consist of one or multiple materials or a multilayered structure). These electrodes may be configured to generate a voltage scaling with the ion concentration to be determined. The voltage at an ion-selective electrode may be compared to a reference electrode arranged to be in contact with a constant concentration of the particular ionic species of interest (but otherwise subject to the same operating conditions as the sensing electrode layer).

As an example, in order to detect a concentration of chloride ions in sweat, two silver (Ag) electrodes with a silver chloride (AgCl) surface layer may be used. One of the electrodes may be modified with a coating containing a fixed concentration of chloride ions (e.g., a polyhydroxyethylmethacrylate (pHEMA) gel) to provide the reference electrode, such that the voltage of the reference electrode is independent of the ionic content of the sweat. The voltage difference between the two electrodes will thus depend on the chloride concentration in the fluid. In particular, the voltage difference may be given by:

V=59 mV log [Cl⁻]+offset Eqn. 1

The sensor can be made sensitive to other ions by adding a semi-permeable membrane (e.g., polyvinyl chloride with certain additives tuned to target ionic species) on top of the other (non-reference) Ag/AgCl electrode. Other examples are possible as well.

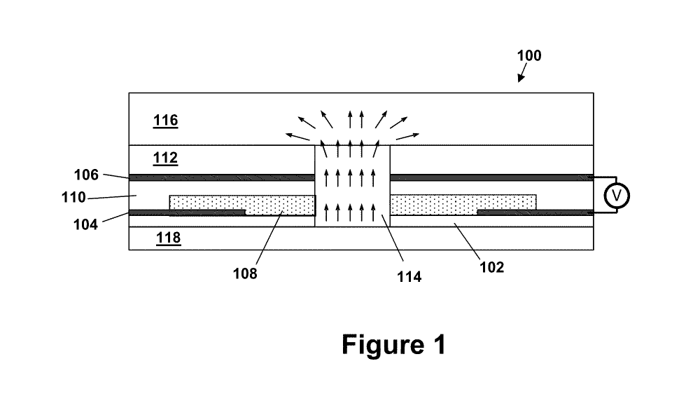

FIG. 1 illustrates an example monitoring apparatus 100, in accordance with some embodiments. The monitoring apparatus 100 may be configured to monitor any type of fluid, including, for example, bodily fluids, such as sweat. In some embodiments, the monitoring apparatus 100 may be connectable to a body (e.g., to skin 118 on the body) of a subject.

As shown, the monitoring apparatus 100 includes a substrate 102, a first electrode 104 and a second electrode 106. The first electrode 104 may serve as a reference electrode. To this end, at least a portion of the first electrode 104 may be in contact with a first material 108 configured to provide a constant ionic concentration.

The first electrode 104 and the first material 108 may together form a first electrode layer. As shown, the first electrode layer is formed on the substrate 102 and is separated from the second electrode 106 by a first insulating layer 110. While only one first insulating layer 110 is shown, in some embodiments more first insulating layers are possible. Further, as shown, the second electrode 106 may be covered by a second insulating layer 112. The first electrode 104, first material 108, first insulating layer 110, second electrode 106, and second insulating layer 112 may form a multilayer structure, as shown.

A flow channel 114 may be provided through the multilayer structure, as shown, to provide a flow path for the fluid to be monitored. In some embodiments, the flow channel 114 may be dimensioned so that the fluid fills the flow channel 114 due to capillary action. Further, as shown, the flow channel 114 may extend substantially perpendicularly to the multilayer structure of the monitoring apparatus 100.

As shown, the first material 108 may form a portion of a sidewall of the flow channel 114. The first electrode 104 is not in contact with the flow channel 114 (nor, accordingly, the fluid), so that the first electrode 104 may serve as a reference electrode.

As shown, the second electrode 106 also forms a portion of the sidewall of the flow channel 114. Because the second electrode 106 is in contact with the flow channel 114 (and, accordingly, the fluid), the second electrode 106 may serve as an ion-sensitive electrode.

In some embodiments, the monitoring apparatus 100 may be configured for detection of a concentration of chloride ions in sweat. In these embodiments, the first electrode 104 and the second electrode 106 may each be formed of silver (Ag) coated with silver chloride (AgCl), as described above. The first material 108 may be any material that provides a fixed concentration of chloride ions in use, such as, for example, Poly(2-hydroxyethyl methacrylate) (pHEMA) gel. The first electrode 104, the second electrode 106, and the first material 108 may take other forms as well.

In these embodiments, a concentration of chloride ions may be determined by measuring a voltage difference between the first electrode 104 and the second electrode 106, as shown. Because the voltage difference scales with the concentration of chloride ions, as described above, the concentration of chloride ions may be determined from the voltage difference.

In some embodiments, an absorbent layer 116 may be positioned on top of the flow channel 114. The absorbent layer 116 may be formed of, for example, a hygroscopic material. Other materials are possible as well. The absorbent layer 116 may be configured to induce a flow of the fluid in the flow channel 114, such that the fluid is substantially continuously replenished in the flow channel 114. In some applications, the absorbent layer 116 could be embedded in a textile. For example, in embodiments where the fluid to be monitored is a bodily fluid (e.g., sweat), the absorbent layer 114 could be included as part of clothing, such as sports clothing.

Alternatively, in other embodiments, an absorbent material 116 may not be used. In these embodiments, the flow channel 114 may be exposed to air, such that evaporation will facilitate a substantially continuous flow of the fluid in the flow channel 114.

As the example monitoring apparatus is formed of a multilayer structure, the monitoring apparatus may be fabricated using various printing and/or deposition techniques. Each of the electrode, insulating, and/or absorbent layers may be relatively thin. For example, the second electrode may have a thickness on the order of, for instance, 1 micron, as this thickness is sufficient to enable the ionic concentration to be measured. Other examples are possible as well. Such relatively thin layers allow for the formation of the monitoring apparatus in a thin, flexible patch, which may be worn by a subject without discomfort, and/or the integration of the monitoring apparatus into a textile, as described above.

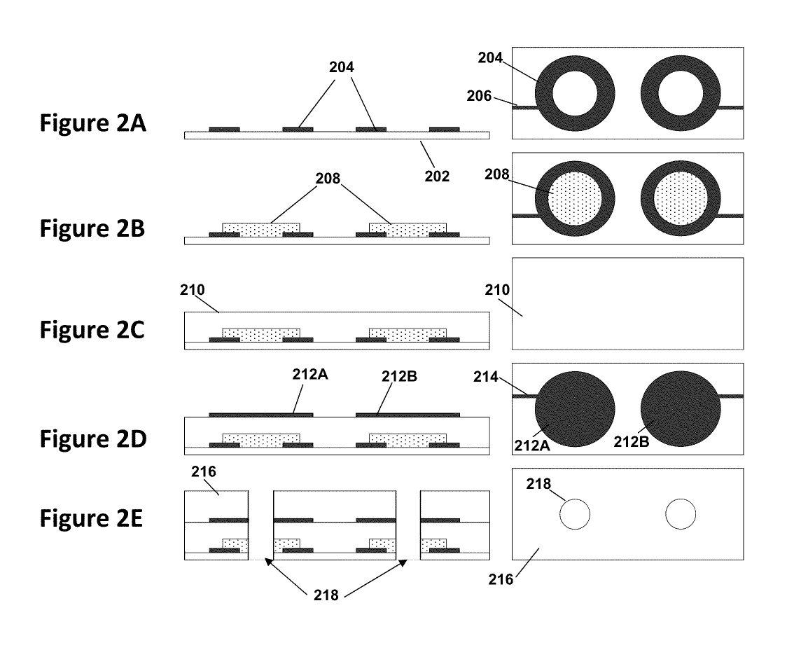

FIG. 2 illustrates a method of fabricating a monitoring apparatus, in accordance with some embodiments. FIG. 2 illustrates formation of a multilayer structure having a plurality of flow channels and shows a series of stages of formation of the multilayer structure in section view on the left and in plan view on the right.

As shown in FIG. 2A, the method begins with providing a substrate 202 and forming first electrodes 204 on the substrate 202. The substrate 202 may take any suitable form, and may be formed of, for example, silicon, glass, and/or foil. Other substrate materials are possible as well. In some embodiments, the substrate 202 itself take the form of a multilayer structure. Other substrates are possible as well.

In applications where a fluid to be monitored by the monitoring apparatus is a bodily fluid (e.g., sweat), the substrate 202 may be configured to contact a subject's skin. To this end, the substrate 202 may be formed of and/or coated with a suitable, non-irritating material. Alternatively or additionally, the substrate 202 may include an adhesive configured to attach the monitoring apparatus to the skin and/or a porous material configured to provide a route through which the bodily fluid may flow into the monitoring apparatus. Such an adhesive and/or porous material may be applied to the substrate 202 following fabrication.

In some embodiments, the substrate 202 itself may not contact the skin, but rather may be positioned on an external side of the monitoring apparatus.

The first electrodes 204 may be formed and patterned into any number of shapes and patterns using, for example, sputtering and/or evaporation techniques followed by photolithography, inkjet printing, and/or screen printing techniques. The first electrodes 204 may be formed of any number of materials suitable for use in electrochemistry, including, for example, platinum, gold, carbon, diamond, and silver. Other materials are possible as well.

The first electrodes 204 may take any number of shapes, so long as the first electrodes 204 do not directly form a sidewall of the flow channel described below. In some embodiments, such as that shown, the first electrodes 204 may be ring-shaped. Other shapes are possible as well.

In some embodiments, each of the first electrodes 204 may be electrically coupled to a conductive track 206 so as to allow electrical connection to the first electrodes 204.

As shown in FIG. 2B, the first electrodes 204 may be at least partially covered by a first material 208. The first material 206 may maintain a constant chloride concentration after exposure to water. For example, the first material 208 may be pHEMA. Other first materials are possible as well. The first material 208 may be patterned using any number of techniques including, for example, printing techniques. As shown, the first material 208 may at least partially cover the first electrodes 204 and may also at least partially cover the substrate 202 in an area of the substrate 202 in which the flow channels will be formed, as described below. In general, an area of the first material 208 may not wholly overlap with an area of the first electrodes 204.

The first electrode 202 and the first material 208 may form a first electrode layer.

The first electrode layer may be covered by a first insulating layer 210, as shown in FIG. 2C. The first insulating layer 210 may be formed from any material or combination of materials that is compatible with the rest of the monitoring apparatus and suitable for the intended application. For example, in some embodiments the first insulating layer 210 may be formed of silicon dioxide (SiO₂), silicon nitride (Si₃N₄), or a layer of printable material (e.g., polyimide or an insulating epoxy). The first insulating layer 210 may itself be have a multilayer structure.

Second electrodes 212A, 212B may then be formed, as shown in FIG. 2D. In some embodiments, each of the second electrodes 212A, 212B may be formed of silver (Ag) coated with silver chloride (AgCl), as described above. The second electrodes 212A, 212B may be formed in any of the manners described above for the first electrodes 204.

As shown, the second electrodes 212A, 212B are patterned to be aligned above the first material 206 and the first electrodes 204. In other embodiments, however, the second electrodes 212A, 212B may be formed and patterned into any number of shapes and patterns. In general, the second electrodes 212A, 212B may be positioned so as to form a portion of the sidewall of the flow channel described below.

Each of the second electrodes 212A, 212B may be provided with a conductive track 214 so as to allow electrical connection to the second electrodes 212A, 212B.

A second insulating layer 216 is then deposited and flow channels 218 are formed through the multilayer structure, as shown in FIG. 2E. The flow channels 218 may be formed using any of a number of techniques, such as, for example, laser ablation, dry etching techniques, and/or simple mechanical punching (depending as appropriate on material and channel size). Within the flow channels 218, a portion of the second electrodes 214A, 214B is exposed, forming a portion of the sidewall of the flow channels 218. Similarly, within the flow channels 218, a portion of the first material 106 is exposed, forming a portion of the sidewall of the flow channels 218. The first electrodes 204 are not exposed within the flow channels 218 and do not form any portion of the sidewall of the flow channels 218. In this manner, the first electrodes 204 may serve as reference electrodes.

As mentioned above, the multilayer structure formed of the first electrodes 204, first material 208, first insulating layer 210, second electrodes 212A, 212B, and second insulating layer 216 may be relatively very thin and flexible, so as to be easily worn by a subject without discomfort. Nevertheless, the exact layer thickness for each layer may depend on the materials used and the desired apparatus characteristics (e.g., strength, rigidity, etc.). For example, to provide a relatively thin structure the various layers may have thicknesses of the order of a few hundred microns or less (although thicker layers may be used in some embodiments if desired). The layers could be much thinner in some embodiments, and may for instance be in the range of about 10 nm to several hundred microns. Other thicknesses are possible as well.

A width (e.g., diameter) of the flow channels 218 may similarly be of the order of 10 nm or so to a few hundred microns. The width of flow channels 218 may depend on the application and the type of fluid being monitored. For example, in embodiments where the fluid is sweat, if the monitoring apparatus is designed for use with an athlete where a great deal of sweat is expected, the flow channels 218 may be relatively wide, whereas if the monitoring apparatus is designed for use with an elderly patient who would not be expected to sweat as much, the flow channels 218 may be relatively less wide. In some embodiments, the diameter of the flow channels 218 could be tailored to a sweat rate for the given subject and thus could differ for different subjects.

While not shown, in some embodiments vertical interconnects may be fabricated through the first and second insulating layers 210, 216 to make an electrical connection to the conducting paths 214, 206, respectively. A voltage difference between the first electrodes 204 and the second electrodes 214A, 214B may then be read from a readout connected to the vertical interconnects.

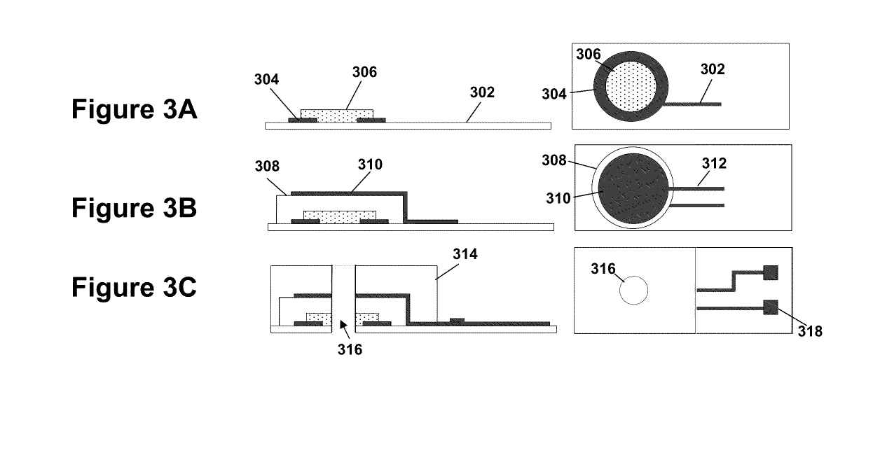

FIG. 3 illustrates another example method of fabricating a monitoring apparatus, in accordance with some embodiments. In particular, FIG. 3 illustrates an alternative way to electrically connect to the electrodes.

As shown in FIG. 3A, a substrate 302 may be provided, a first electrode 304 may be formed on the substrate 302, and a conductive track 306 may be electrically coupled to the first electrode 304 so as to allow electrical connection to the first electrode 304.

The first electrode 304 may be partially coated in a first material 308, which may take any of the forms described above for the first material 308 in connection with FIG. 2. In general, the first material 308 may provide a constant ionic concentration.

A first insulating layer 310 may be formed over the first material 308, as shown in FIG. 3C. As shown, the first insulating layer 310 may be pattern so as to expose a portion of the substrate 302 away from the first electrode 304 (and the intended area of the flow channel, described below). For example, the first insulating layer 310 may be formed as a disk shape. Other shapes and patterns are possible as well.

The second electrode 310 may then be patterned on top of the first insulating layer, as shown, with the conductive tracks 302, 312 extending over the edge of first insulating layer 310 and connecting to and continuing on the substrate 302.

The second electrode 310 may then be covered by a second insulating layer 108, leaving the substrate 302 and ends of conductive tracks 302, 312 exposed, as shown in FIG. 3C. Flow channel 316 is formed through the multilayer structure, as described above. On the substrate 302, additional circuitry 318 may be formed. The additional circuitry 318 may include, for example, contacts to allow for connection of other readout circuitry that can measure voltages or currents, readout and/or analysis circuitry, and/or memory and/or wireless communication circuitry. Conveniently, all the steps in the fabrication process may be performed by printing processes or standard (photo)lithography methods as known to reduce the manufacturing costs. In some embodiments, the substrate 302 may be biodegradable. In this way, the monitoring apparatus may be a disposable item.

It will be appreciated that the flow channels described above may provide a flow path which is substantially perpendicular to the layers of the multilayer structure. This geometry may allow a simple and inexpensive fabrication process that can benefit from various multilayer deposition and patterning techniques, such as various printing techniques. The multilayer structure may, for example, be fabricated by aligning the various electrode layers at a known location and then forming a flow channel through the layers at that location. As only a simple linear channel perpendicular to the layers is needed the channel may, as described above, be formed in a straightforward ablation, etching or punching step.

It will further be appreciated, however, that other geometries would be possible and the flow channel could, if desired, be constructed to run in some other direction and/or to change direction within the multilayer structure, for instance by using sacrificial material to define the flow path during fabrication with subsequent etching or other such techniques. Other geometries are therefore possible as well.

While the above examples focused on a monitoring apparatus in which the second electrode was configured to sense chloride, in other embodiments other ion-selective electrodes may be used and other ion-levels may be determined. For example, a second electrode formed of iridium oxide may be used to monitor a pH level. Other examples are possible as well.

In some embodiments, multiple different sensing electrodes may be used to provide an apparatus capable of monitoring for multiple different analytes. The multilayer structure may thus be readily extended by using the same general fabrication techniques to provide a plurality of electrodes for monitoring a plurality of different species. In some embodiments, the monitoring apparatus may include a plurality of ion-selective electrode layers sensitive to a variety of different target ionic species.

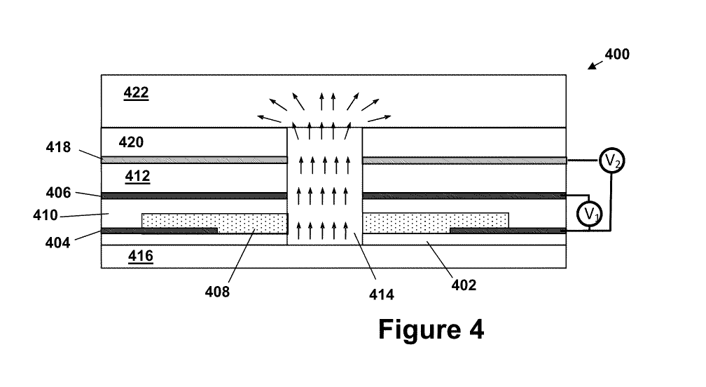

FIG. 4 illustrates an example monitoring apparatus 400 including a plurality of different ion-sensitive electrodes 406, 412, in accordance with some embodiments. As shown, the monitoring apparatus 400 includes a substrate 402, a first electrode 404, a first material 408, a second electrode 406, a first insulating material 410, and a second insulating material 412, similar to the monitoring apparatus 100 described above in connection with FIG. 1. The first material 408 and the second electrode 406 each form a portion of a sidewall of a flow channel 414, as shown, and as described above. In some embodiments, the monitoring apparatus 400 may be configured to contact skin 416 of a subject, as shown. Other applications are possible as well.

Further, as shown, the monitoring apparatus 400 includes a third electrode 418. The third electrode 418 may be formed of a different material than the second electrode 406, such that the third electrode 418 is configured to sense a different analyte. For example, in embodiments where the second electrode 406 is formed of Ag/AgCl and is sensitive to chloride ions, the third electrode 418 may be formed of iridium oxide and may be sensitive to a hydrogen concentration (e.g., pH). A third insulating layer 420 may be formed on the third electrode 418.

A first voltage, V₁, between the first and second electrodes 404, 406 may be used to determine a chloride concentration in a fluid passing through the fluid channel 414, as described above. Further, a second voltage, v2, between the second and third electrodes 406, 418 may be used to determine a hydrogen concentration in the fluid. In this manner, the monitoring apparatus 400 may monitor both a pH level and a chloride level in a substantially continuous and/or real-time manner.

In other embodiments, ion-selective electrodes may alternatively be formed by adding a semipermeable membrane in contact with a suitable electrode, such as a Ag/AgCl electrode. For example, a polyvinylchloride (PVC) membrane with a molecular receptor (e.g., valinomycin, calixarene) may serve to render monitoring apparatus selective to particular analytes (e.g., potassium, sodium, respectively). A variety of ion-selective electrodes for various ions can be formed by changing the composition of the membrane.

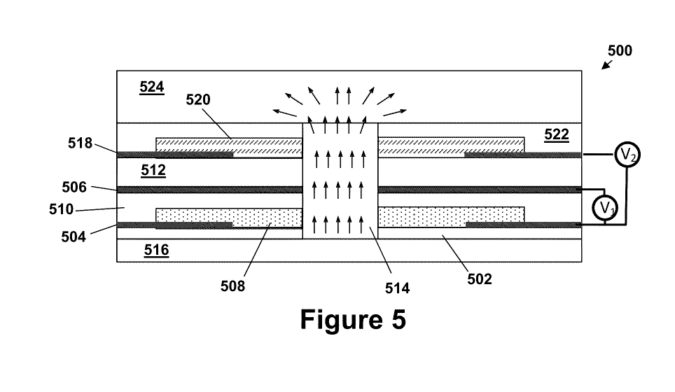

FIG. 5 illustrates a monitoring apparatus 500 similar to that described above in connection with FIG. 4. In particular, the monitoring apparatus 500 includes a substrate 502, a first electrode 504, a second electrode 506, a first material 508, a first insulating material 510, a second insulating material 512, and a flow channel 514. The monitoring apparatus 500 may be configured to connect to a subject's skin 516, as shown.

The monitoring apparatus 500 further includes a third electrode 518. However, as shown, the monitoring apparatus 500 further includes a membrane 520 formed over the third electrode 518. The membrane 520 may be formed of, for example, a suitable membrane material, such as PVC with a suitable molecular receptor. As a result, the voltage V₁ will scale with chloride concentration and the voltage V₂ will scale with a different target ion concentration, such as potassium or sodium.

It will therefore be understood that at least one ion-selective electrode layer may comprise an electrode formed from an electrode material sensitive to a target ionic species where the electrode material itself forms said part of the sidewall of the flow channel, such as the second electrode 506. Additionally or alternatively, at least one ion-selective electrode layer may comprise an electrode in contact with an ion-selective membrane material where it is the ion-selective membrane material (and not the electrode material directly) that forms part of the sidewall of the flow channel, such as the third electrode 518.

Thus, FIGS. 4 and 5 illustrate providing electrodes with different sensing capability within a single flow channel. In other embodiments, a monitoring apparatus may include a plurality of flow channels in which at least some flow channels may have different electrode layers to one another so as to provide different sensing capability. For instance, referring back to FIG. 2, the second electrodes 212A formed for one flow channel 218 may be different to the second electrode 214B formed for a different flow channel 218. For example, second electrode 212A could be an Ag/AgCl electrode to provide sensing of chloride concentration, whereas second electrode 214B could be Iridium oxide to provide sensing of pH. Other examples are possible as well. In operation, both flow channels may experience a continual flow of fluid and thus substantially continuous simultaneous sensing may be provided.

Some compounds of sweat, for instance, lactate, glucose or narcotics or prescribed substances or indicators thereof can be detected by converting the target into an electrochemically active product by an enzyme. As an example, lactate can be converted into pyruvate and hydrogen peroxide by the enzyme lactate oxidase. The hydrogen peroxide can then be detected by a platinum electrode. Similarly, glucose can be detected after conversion by the enzyme glucose oxidase. Such detection may be referred to as amperometric detection.

The amperometric detection mechanism differs compared to operation of the ion-selective electrodes discussed previously. In this instance, a voltage is applied between the platinum electrode, referred to as a working electrode, and a reference electrode, while the current flowing between the working electrode and another electrode, the so-called counter electrode, is measured.

Suitable enzymes may be immobilized into a porous substrate (e.g., a nafion or polycarbonate membrane). In this way, reactive layers that act to convert target chemicals into electrochemically active products may be formed and incorporated into a multilayer structure, in accordance with some embodiments.

In some embodiments, therefore, the multilayer structure may include at least one reactive layer that reacts with at least one target chemical, if present, to produce an electrochemically active product. The reactive layer may form part of the sidewall of the flow channel downstream of at least one electrode layer that is monitored for the presence of the active product. As mentioned, the reactive layer may comprise a porous material having an enzyme immobilized therein that reacts with a target chemical.

The sensing electrodes and reactive layer may be readily incorporated as part of the flow channel in the multilayer structure as described herein. Such a configuration allows for the continuous and real-time determination of a number of additional components of sweat, such as lactate, and thus further extends of the capabilities of the apparatus according to embodiments of the present invention. The reactive layer and corresponding sensing electrodes (i.e., working and counter electrodes), may be used in addition to or instead of the ion-selective electrode layers discussed previously.

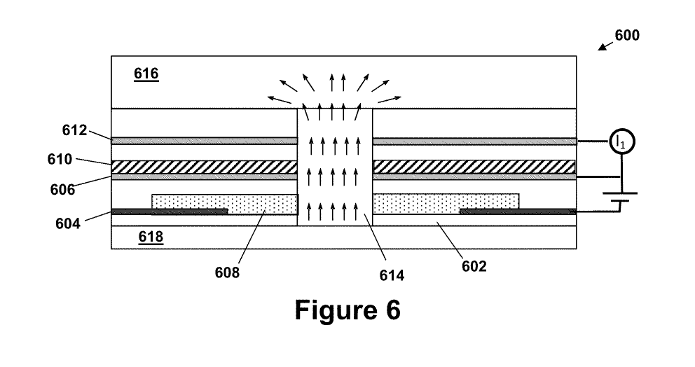

FIG. 6 illustrates an example monitoring apparatus 600 including a reactive layer 610 and sensing electrodes 606, 612, in accordance with some embodiments.

As shown, the monitoring apparatus 600 comprises a reference electrode layer formed from a Ag/AgCl electrode 604 and pHEMA gel 608, as described above. Further, as shown, the monitoring apparatus 600 includes sensing electrodes 606 and 612 which, in this example, may be platinum electrodes or other commonly used materials in electrochemistry, such as gold, carbon or diamond. Sensing electrodes 606, 612 may be are arranged to form part of the sidewall of the flow channel 614, as shown.

The monitoring apparatus 600 also includes the reactive layer 610, as described above. In some embodiments, the reactive layer 610 may comprise a porous membrane layer of PVC having enzymes immobilised therein, as discussed above. As sweat flows through the flow channel 314, at least some sweat will interact with the reactive layer 610. The reactive layer 610 may, for example, be used to determine a lactate concentration of the sweat flowing through the flow channel 614. Other examples are possible as well.

As shown, the reactive layer 610 may be fabricated directly on top of a sensing electrode 606. Electrode 606 is formed upstream in the flow path and thus both sensing electrodes 606, 612 are positioned at suitable parts of the flow path.

Alternatively, in other embodiments the reactive layer 610 may be arranged downstream in the flow path of both sensing electrodes 606, 612 and possibly separated from sensing electrode 606 by, for example, an insulation layer (not shown). In the event of the presence of the target chemical the electrochemically active products will thus be introduced into the rest of the flow path of the flow channel 614 and be detected upstream. The configuration used (in terms of location of the reactive layer 610) may depend on the nature of the enzymes and membranes (e.g., whether the enzyme is compatible with a metal electrode).

In operation, a voltage may be applied between the reference electrode 604 and the working electrode (e.g., sensing electrode 606), and the current between the working electrode and a counter-electrode (e.g., sensing electrode 612) may be measured.

The fabrication scheme of the multilayer structure described in effect provides simple building blocks that can be stacked on top of each other to add more parameters to be monitored as desired. The resulting monitoring apparatus is therefore highly tunable for specific applications.

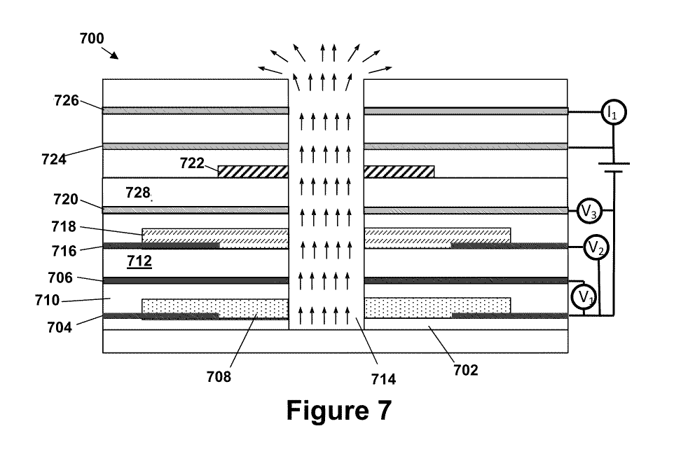

FIG. 7 illustrates an example monitoring apparatus 700 having multiple sensing capability, in accordance with some embodiments. In particular, the monitoring apparatus 700 may take the form of a sweat monitoring apparatus suitable for the substantially continuous monitoring of pH, lactate, chloride, and sodium levels.

As shown, the monitoring apparatus 700 includes a substrate 702, a reference electrode 702 (e.g., an Ag/AgCl electrode) and a first material 706 (e.g., a pHEMA gel 708). The monitoring apparatus 700 further includes a first sensing electrode 704 (e.g., an Ag/AgCl electrode) configured to monitor chloride ion concentration. The monitoring apparatus 700 further includes a second sensing electrode 716 (e.g., an Ag/AgCl electrode) having an ion sensitive membrane 718, as described above, configured for detecting a sodium ion concentration. A third sensing electrode 720 (e.g., an iridium oxide electrode) may be configured for detecting pH level.

A reactive layer 722 may take the form of a porous membrane having an enzyme for converting lactate into an active product for detection by first and second sensing electrodes 704, 716. In some embodiments, the reactive layer 722 may be positioned downstream of the sensing electrodes 704, 716, as described above. A potential difference may thus be applied between a working electrode 724 and reference electrode 704, as shown.

FIG. 7 also shows that a flow channel 714 that is open to air. In alternative embodiments, an absorbent could be used, as described above.

The voltages V₁, V₂, and V₃ scale with chloride, sodium and pH, respectively, while the current I₁ scales with lactate concentration. Monitoring these parameters may be desirable for, for instance, early detection of dehydration or fatigue in sports.

It will of course be understood that the order of electrodes is not fixed and the electrodes could be arranged in a different order if desired. The only electrode required for each analyte is the reference electrode, while more functionality can be implemented by adding more electrodes depending on the application. As mentioned above, at least some of the different sensing functions may be implemented by using different electrodes for different flow channels.

It will of course be appreciated that in order for the disclosed monitoring apparatuses to function correctly the fluid to be monitored should flow substantially continually so as to replenish the flow channel(s). In embodiments where the fluid to monitored is sweat, if a subject is performing reasonable strenuous activity, such as during sports, or is in an environment of elevated temperature, the sweat produced by the subject may be sufficient to fill the flow channels in the multilayer patch apparatus by capillary forces. In some applications, however it may be wished to monitor the sweat of a subject in situations where the subject is not sweating significantly. For example hospitalized elderly patients do not tend to sweat much, but it may be useful to monitor the sweat content of such patients to spot signs of dehydration, for example. If the subject does not produce significant sweat, the flow channel(s) will not be filled with sweat and/or may dry out. In some embodiments, therefore the monitoring apparatus may comprise at least one layer of a material capable of inducing sweating in the subject.

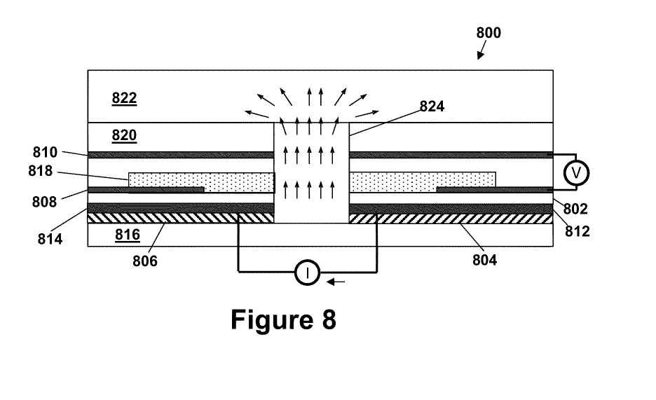

FIG. 8 illustrates an example monitoring apparatus 800 including a layer 804, 806 for inducing sweating, in accordance with some embodiments. As shown, the monitoring apparatus 800 includes a substrate 802, a reference electrode 808, and a sensing electrode 810. Further, the monitoring apparatus includes additional electrodes 812, 814, at least one of which is modified with a hydrogel 804 containing a chemical such as pilocarpine which can induce sweating. In use, the hydrogel 804 may be arranged in direct contact with the skin 816 of the subject. Hydrogel 806 containing inert salt sodium nitrate is also provided adjacent one of the electrodes 812, 814 and also arranged in contact with the skin 816. Pilocarpine is a substance that can induce sweating when it is forced into the skin. This can be achieved by applying a small current of ˜1 mA between the electrodes for several minutes. The pilocarpine is forced into the skin by iontophoresis, after which the sweating commences. The hydrogel 806 containing sodium nitrate sustains the electric current when sweating is induced.

The disclosed monitoring apparatuses may be used in in a range of applications to provide substantially continuous, real-time monitoring. In embodiments where the monitoring apparatus takes the form of a patch (e.g., to monitor sweat), the patch may be reusable and/or may be integrated into clothing. The disclosed monitoring apparatuses may find application in the field of sports or physical training, for instance providing feedback for personalized uptake of water and salt by athletes and/or for monitoring for use of prescribed substances. Further, the sweat monitoring apparatus could be used in medical fields, for instance for continuous point-of-care dehydration monitoring for elderly and ill people, at home or in hospitals. Whilst the sweat monitoring apparatus is principally useful for monitoring human subjects, there may be applications where it is useful to monitor the sweat of an animal that produces sweat, for instance in the training of race horses or the like.

As mentioned previously, the multilayer structure, especially when used with a flow path geometry which is substantially perpendicular to the layers, allows low cost fabrication. All the layers in the fabrication process can be printed to reduce costs, and thus the sweat monitoring patch could be disposable.

In some applications, as described above, the sweat monitoring apparatus may additionally be used for the single determination of other analytes in sweat that require antibodies for detection, such as drugs of abuse, hormones or disease markers. In these applications, the sweat monitoring apparatus may comprise at least one sensing layer which includes a sensing layer that reacts to the presence of a target chemical to undergo a physical change that can be detected, as described above. For instance, the physical change may comprise analytes binding to antibodies to change an impedance of an electrode. Other examples are possible as well.

To this end, the monitoring apparatus may therefore comprise a porous material in contact with at least one electrode treated with a sensing material that reacts with a target chemical, where the porous material forms part of the sidewall of the flow channel. In use the sweat will flow through the flow channel and some sweat will be drawn into the porous material. If the target analyte is present, the sensing material will react and the presence of the target analyte can be detected.

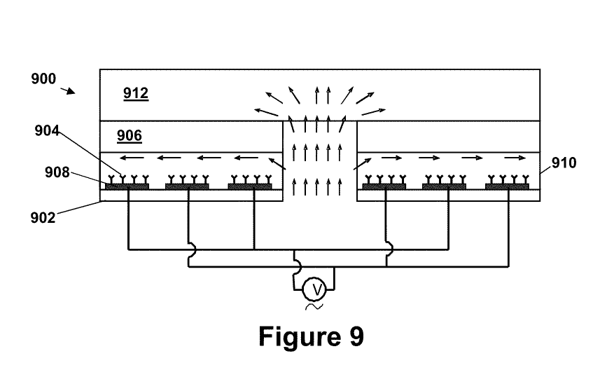

FIG. 9 illustrates an example monitoring apparatus 900 including a sensing layer 902 that reacts with target chemicals to provide a detectable change in physical properties, in accordance with some embodiments. As shown, the monitoring apparatus 900 includes a layer 902 that includes electrodes 908 modified with a sensing material 904. In some embodiments, the layer 902 may comprise the top layer (other than an absorbent, if used) or an insulating layer of any of the multilayer stacks described above. The electrodes 908 can be of any shape or material, and may be gold in an interdigitated array design, as is shown in FIG. 9.

The electrodes 908 may be coated or otherwise treated with sensing material 904, which may comprise suitable antibodies for the target chemical. The electrodes 908 and sensing material 904 are covered by an absorbent or other porous layer 910, such as nafion. The porous layer 910 forms part of the sidewall of the flow channel through the multilayer structure and thus, in use, part of the sweat flowing through the channel is absorbed in this layer.

The analyte, if present, reacts with the sensing material 904 to form a complex, which is detected, for instance, by impedance spectroscopy. In this technique, an alternating voltage is applied between pairs of electrodes and the impedance is determined.

As the analyte-antibody complex formation is nearly irreversible, the disclosed monitoring apparatus may only be used for a single detection. Nevertheless, the disclosed monitoring apparatus provides substantially continuous and real-time monitoring and provides more functionality to the multilayer monitoring capability.

The embodiments described above with reference to FIGS. 1-9 have principally been described with reference to embodiments in which the fluid to be measured is sweat. However, the disclosed monitoring apparatuses may be used to measure other fluids, including other bodily fluids.

For example, a sensor according to any of the embodiments described above could be used for wound monitoring to monitor the content of fluid produced by the wound. The multilayer apparatus could be incorporated as part of a wound dressing, for example. The absorbent material could form part of the dressing. In use, fluid produced by the wound would be drawn through the flow channels in the same way as discussed previously and thus can be analyzed using any of the sensing techniques discussed above, which are applicable to monitoring the condition of the wound. Such a wound monitoring apparatus may therefore be able to provide continuous monitoring of the state of the wound by monitoring the fluid for analytes indicative of the state of the wound. This may reduce the need for continual inspection and/or replacement of monitoring apparatus, which may hinder recovery but provide early warning of any possible problems.

Likewise embodiments of the invention may be used to monitor saliva. An apparatus such as that shown in FIG. 1, for example, could be configured to be retained in a subject's mouth, for instance as a patch applied to the inside of the mouth. In this embodiment, as the patch may generally be immersed in the fluid to be monitored, absorbent material may be used to draw the fluid through the flow channel(s) as shown, but the outside of the absorbent material may be sealed, in use, from the environment. In other words, the absorbent material may be in fluid contact with the environment only through the flow channel(s). Further, as the apparatus may be usually immersed in the fluid to be monitored, the flow channel inlet could be arranged on the outer surface of the monitoring apparatus. In other words, rather than arranging the inlet to the flow channel adjacent the body, the inlet could be located on the opposite surface of the apparatus to that which is in contact with the body. In this way saliva may be drawn through the flow channel, with the sealed absorbed providing a continual drawing of saliva through the flow channel. Again, any of the measurement techniques described above may be implemented to monitor for analytes of interest in saliva.

Thus embodiments of the invention relate to fluid monitoring apparatuses that may be contactable with a surface at which a fluid of interest may be produced. The apparatus may be attachable to such a surface, which may be a surface of the body of a subject. In other embodiments the apparatus may be deployed so as to be immersed in a fluid of interest in use.

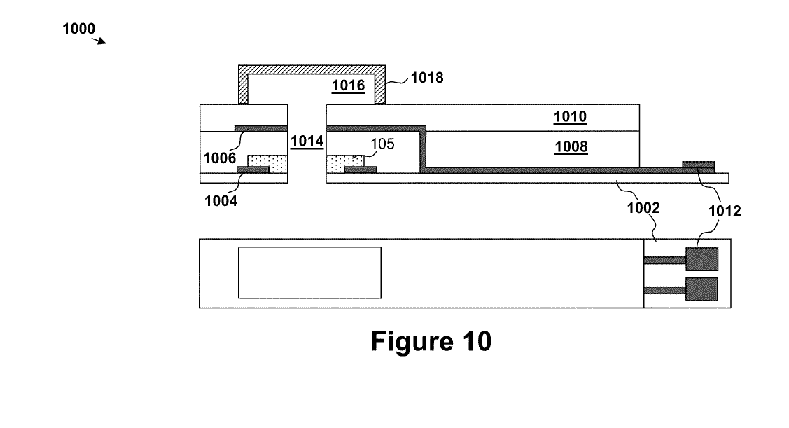

FIG. 10 shows, in side and plan views, an additional embodiment of a monitoring apparatus 1000 suitable for being immersed in, or brought into contact with, a fluid of interest by a user. As shown, the monitoring apparatus 1000 is an elongate apparatus with a sensing portion at one end including the flow channel 1014 and electrode layers 1004, 1106 and a handle portion at the opposite end. In the apparatus shown in FIG. 10, the substrate 1002 and insulating layers 1008 and 1010 may be elongate to provide the sensing and handle portion, but in other embodiments the multilayer apparatus could be mounted to a suitable handle.

Readout circuitry/contacts 1012 may be located at the handle end of the apparatus which is remote from the flow channel 1014.

One end of the flow channel 1014 is adjacent an absorbent material 1016 as described previously, but in this embodiment the absorbent 1016 is located adjacent the substrate 1002. The absorbent material 1016 is contained within a sealing material 1018 which is fluid impermeable, such that the only flow path for fluid in use is via the flow channel 1014. In use, a user may therefore hold the handle end of the apparatus 1000 such that the sensing portion is immersed in, or in contact with, the fluid of interest. For example the apparatus could form a spatula-like apparatus to be placed in the mouth of a subject to monitor saliva content over a period of time. In use the fluid, e.g., saliva, may be drawn through the flow channel 1014 and analysed as described previously.

Optionally, there may be an additional absorber in contact with the inlet to the flow channel 1014. This additional absorber may be useful in situations where there is not much fluid in the environment (e.g., when a subject has a relatively dry mouth). The absorber may draw fluid to the inlet. Once absorber is relatively saturated capillary action, and the action of the absorber, will draw fluid through the flow channel.

1. An apparatus, comprising:
a multilayer structure comprising:
(i) a first electrode layer comprising a first electrode and a first material,
(ii) a second electrode layer comprising a second electrode, and
(iii) an insulating layer positioned between the first electrode layer and the second electrode layer; and
a flow channel configured to receive a substantially continuous flow of fluid, wherein each of the first material and the second electrode layer form a portion of a sidewall of the flow channel.

2. The apparatus of claim 1, wherein the flow channel extends substantially perpendicularly to the multilayer structure.

3. The apparatus of claim 1, wherein:
the first electrode comprises a reference electrode;
the first material comprises a material having a substantially constant ionic concentration; and
the second electrode comprises an sensing electrode configured to sense an analyte.

4. The apparatus of claim 3, wherein a voltage difference between the reference electrode and the sensing electrode is proportional to a concentration of the analyte in the fluid.

5. The apparatus of claim 1, further comprising a reactive layer configured to convert a target chemical in the fluid into an analyte, wherein the second electrode comprises a sensing electrode layer configured to sense an analyte.

6. The apparatus of claim 5, wherein the reactive layer is positioned in a downstream direction from the sensing electrode.

7. The apparatus of claim 5, wherein the reactive layer forms a portion of the sidewall of the flow channel.

8. The apparatus of claim 5, wherein the reactive layer comprises a porous material having an enzyme immobilized therein that reacts with the target chemical in the fluid to produce the analyte.

9. The apparatus of claim 1, further comprising an absorbing layer adjacent to the flow channel, wherein the absorbing layer is configured to induce the substantially continuous flow of fluid.

10. The apparatus of claim 1, wherein the fluid is sweat, the apparatus further comprising at least one layer of material configured to induce sweating.

11. The apparatus of claim 1, further comprising at least one of: electrical circuitry configured to substantially continuously read out sensing data indicating a voltage difference between the first electrode and the second electrode, electrical circuitry configured to store the sensing data, electrical circuitry configured to transmit the sensing data, and electrical circuitry configured to process the sensing data to determine a concentration of an analyte in the fluid.

12. The apparatus of claim 1, further comprising:
a third electrode layer comprising a third electrode, wherein:
the second electrode comprises a first sensing electrode configured to sense a first analyte in the fluid; and
the third electrode comprises a second sensing electrode configured to sense a second analyte in the fluid.

13. The apparatus of claim 1, wherein the apparatus is integrated with a textile.

14. The apparatus of claim 1, wherein the apparatus is integrated with a patch.

15. An apparatus, comprising:
a multilayer structure comprising a plurality of flow channels configured to receive a substantially continuous flow of fluid, wherein a sidewall of each flow channel is formed by:
(i) a first electrode layer comprising a first electrode and a first material, wherein the first material forms the sidewall;
(ii) a second electrode layer comprising a second electrode; and
(iii) an insulating layer positioned between the first electrode layer and the second electrode layer.

16. The apparatus of claim 15, wherein:
the plurality of flow channels comprises a first flow channel and a second flow channel;
the second electrode of the first flow channel is configured to sense a first anaylte in the fluid; and
the second electrode of the second flow channel is configured to sense a second analyte in the fluid, wherein the second analyte differs from the first analyte.

17. The apparatus of claim 15, wherein the flow channel extends substantially perpendicularly to the multilayer structure.

18. A method, comprising:
providing a monitoring apparatus comprising:
(i) a first electrode layer comprising a first electrode and a first material,
(ii) a second electrode layer comprising a second electrode, and
(iii) an insulating layer positioned between the first electrode layer and the second electrode layer; and
a flow channel configured to receive a substantially continuous flow of fluid, wherein each of the first material and the second electrode layer form a portion of a sidewall of the flow channel;
inducing the substantially continuous flow of fluid;
measuring a voltage difference between the first electrode and the second electrode; and
based on the voltage difference, determining a concentration of at least one analyte in the fluid.

19. The method of claim 18, wherein the monitoring apparatus further comprises a third electrode layer comprising a third electrode, the method further comprising:
measuring an additional voltage difference between the first electrode and the third electrode; and
based on the additional voltage difference, determining a concentration of at least one additional analyte in the fluid, wherein the at least one additional analyte differs from the at least one analyte.

20. The method of claim 18, wherein the fluid comprises one of sweat and saliva.
